(19) United States
(12) Patent Application Publication
(10) Pub. No.: US 2010/0197635 A1
(43) Pub. Date: Aug. 5, 2010

(54) TREATMENT OF TARDIVE DYSKINESIA WITH PYRIDOXAL 5'-PHOSPHATE

(75) Inventors: Albert Friesen, Winnipeg (CA); Dawson James Reimer, Winnipeg (CA)

Correspondence Address: MERCHANT & GOULD PC, P.O. BOX 2903, MINNEAPOLIS, MN, 55402-0903, US

(73) Assignee: Medicure International Inc.

(21) Appl. No.: 12/602,443

(22) PCT Filed: May 28, 2008
(86) PCT No.: PCT/CA08/01024

§ 371 Date: Apr. 7, 2010

(30) Foreign Application Priority Data
May 28, 2007 (CA) 2590603

(51) Int. Cl. A61K 31/675; C07F 9/12; A61P 25/14

(52) U.S. Cl. 514/89; 546/24

(57) ABSTRACT

This invention relates to treatment of Tardive Dyskinesia through the administration of pyridoxal 5′-phosphate. The treatment may be an oral administration of 100-4000 mg/day, for example, 100-750 mg/day or about 250 mg/day. Also provided is the use of pyridoxal 5′-phosphate for the preparation of a medicament for the treatment of tardive dyskinesia, kits comprising pyridoxal 5′-phosphate for the treatment of tardive dyskinesia, and combination drugs for the treatment of tardive dyskinesia.

### CROSS-REFERENCE TO RELATED APPLICATION

This application claims priority to and the benefit of Canadian Patent Application No. 2,590,603 filed May 28 2007, which is incorporated herein by reference.

### FIELD OF THE INVENTION

This invention relates to treatment of Tardive Dyskinesia through the administration of pyridoxal 5′-phosphate.

### BACKGROUND OF INVENTION

Tardive Dyskinesia (TD) can be characterized as a set of repetitive uncontrollable movements primarily of the facial muscles. The occurrence of TD is directly correlated to the use of antipsychotic medications. Some of the factors associated with the susceptibility to TD are old age, female sex, presence of a major defective disorder, exposure to antipsychotic drugs, use of anticholinergic/antiparkinson drugs and history of extrapyramidal syndrome. Schizophrenic patients usually receive neuroleptics as treatment, which quite often leads to TD. Incidence of TD is greater than 20% in individuals receiving neuroleptics therapy (Bhoopathi and Soares-Weiser, 2006).

The underlying cause of TD is said to be unclear however some researchers theorize that the role of vitamin B6 in the synthesis of various neurotransmitters (including serotonin, dopamine, gamma-aminobutyric acid (GABA) and norepinephrine) may be central to the development of TD (Miodownik et al., 2000).

Animal studies have shown that as vitamin B6 levels increase, the levels of brain 5-HT (serotonin) increased. It is also known that vitamin B6 deficiency may result in reduced concentrations of 5-HT in the brain (Miodownik et al., 2000). Vitamin B6 supplementation may promote adequate neurotransmitter synthesis and may reduce the incidence of TD.

A double-blind, placebo-controlled crossover study conducted among patients with TD induced by the use of schizophrenia medication revealed that administration of 100-400 mg vitamin B6 per day lead to improved scores on the subscales of the Extrapyramidal Symptom Rating Scale, more specifically the parkinsonism and dyskinetic movement scales (Lerner et al., 2001). The conclusion from this study was that administration of pyridoxine in doses of 300 mg/day or greater are required for the treatment of TD.

The mechanism of action of pyridoxine is not clear since plasma P5P levels do not display a linear relationship with perfect positive correlation. It is believed that the role of P5P in decarboxylation of dopa resulting in the production of dopamine as well as formation of other neurotransmitters (gamma-aminobutyric acid, serotonin and melatonin) may be involved in the symptoms of TD (Lerner et al., 2001).

Pyridoxine is absorbed from the digestive tract where it is transported to the liver and oxidized to form pyridoxal. It is then phosphorylated by pyridoxal kinase to form pyridoxal 5′-phosphate (P5P)(Vermaak et al., 1988). Administration of pyridoxine to treat conditions of the previously mentioned disorders has shown to improve healing, however the effects may be limited based on the dose.

Pyridoxine has been known to produce toxicity at high doses, and limits plasma P5P levels and the U.S. National Academy of Sciences has therefore recommended 100 mg as the Tolerable Upper Intake Level (unlikely to pose risks of adverse health effects in healthy people) for pyridoxine (National Academy of Science, 1998). With respect to safety, the main toxicity associated with vitamin B₆ is neurotoxicity. The administration of pyridoxine results in high plasma levels of pyridoxine which can lead to toxic effects such as peripheral neuropathy (Schaumburg et al, 1983). The sensory neuropathy that occurs following administration of large dose levels of oral pyridoxine has been attributed to high circulating concentrations of pyridoxine per se. High concentrations of pyridoxine lead to the inhibition of P5P binding to apoenzymes resulting in decreased levels of plasma P5P (the active form) (Bassler, 1988) and thus greater levels of P5P are achieved by its direct administration.

Pyridoxine treatment of TD has been limited by the toxicity of pyridoxine-administering high dosages of pyridoxine can result in toxicity and side effects.

Typically, with most drugs, increasing the dosage of the drug is desirable, rendering the drug more efficacious. This increase in dosage is mitigated by side effects and toxicity, which typically increase with dosage.

Pyridoxal 5′-phosphate (3-hydroxy-2-methyl-5-[(phosphonooxy)methyl]-4-pyridine-carboxaldehyde, or “P5P”) is a naturally occurring metabolite of pyridoxine and is formed in mammalian cells by phosphorylation and oxidation reactions. A recent evaluation demonstrated that P5P inhibits adenosine triphosphate (ATP) induced calcium ion influx into cells. Results suggest that this action is due to an inhibition of purinergic receptors known as P2 purinoceptors.

P5P can be chemically synthesized in a number of ways, for example, by the action of ATP on pyridoxal, by the action of phosphorus oxychloride on pyridoxal in aqueous solution, and by phosphorylation of pyridoxamine with concentrated phosphoric acid followed by oxidation.

It would be desirable to have a treatment for TD that can be given at relatively high concentrations, with a minimum of side effects or toxicity. It would also be desirable to have a compound that can be administered in conjunction with a neuroleptic that would mitigate the TD effects of said neuroleptic.

### SUMMARY OF THE INVENTION

According to one embodiment of the present invention is a method for the treatment of tardive dyskinesia comprising administration of pyridoxal 5′-phosphate.

According to one aspect, the tardive dyskinesia is a result of use of schizophrenia medication, for example, a neuroleptic.

According to a further aspect, the administration is an oral administration of between 100-4000 mg/day, for example, between 100-750 mg/day, or about 250 mg/day.

According to a further aspect, the administration is provided in combination with a schizophrenia drug, for example, a neuroleptic.

According to a further embodiment of the present invention is provided the use of P5P in the preparation of a medicament for the treatment of tardive dyskinesia.

According to a further aspect, the medicament comprises between 100-4000 mg of pyridoxal 5′-phosphate, for example, between 250-750 mg of pyridoxal 5′-phosphate, or about 250 mg of pyridoxal 5′-phosphate.

According to a further aspect, the medicament also comprises a schizophrenia drug, for example, a neuroleptic.

According to a further embodiment of the present invention is provided the use of pyridoxal 5′-phosphate for the treatment of tardive dyskinesia.

According to a further aspect, the tardive dyskinesia is a result of treatment with a schizophrenia medication, for example, a neuroleptic.

According to a further aspect, the treatment comprises administration of between 100-4000 mg of pyridoxal 5′-phosphate per day, for example, between 100-750 mg/day, or about 250 mg/day.

According to a further aspect, the use further comprises the use of a neuroleptic to treat schizophrenia.

According to a further embodiment of the present invention is provided a kit comprising: (a) a pharmaceutical preparation for oral administration comprising pyridoxal 5′-phosphate; (b) instructions for the administration of said preparation;

wherein said instructions specify that the preparation is to be administered daily to a patient for the treatment of tardive Dyskinesia.

According to a further aspect, the instructions specify that the preparation should be administered in a dosage range of between 100-4000 mg/day, for example, between 250-750 mg/day or about 250 mg/day.

According to a further embodiment of the present invention is provided a pharmaceutical compound comprising pyridoxal 5′-phosphate and a schizophrenia drug.

According to a further aspect, the schizophrenia drug is a neuroleptic.

According to a further embodiment of the present invention is provided the use of pyridoxal 5′-phosphate to mitigate the tardive dyskinesia effects of a schizophrenia drug, for example, a neuroleptic.

### DETAILED DESCRIPTION

The present inventors have surprisingly found that pyridoxal 5′-phosphate is safe and low in side effects at relatively high concentrations, including concentrations previously known to be undesirable for vitamin B6.

The present inventors have found that P5P is thus more tolerable than vitamin B6 and has a much higher dosage requirement to result in toxicity, such as is observed with high doses of vitamin B6. High doses of P5P are recognized as an improvement in treatment.

Even at low dosages or dosages equivalent to vitamin B6 dosages, P5P has been found to be more efficacious than vitamin B6 in treating tardive dyskinesia.

### EXAMPLE 1

Safety and Tolerance of Pyridoxal-5′-Phosphate Enteric-Coated Tablet

A Phase I, single center, single-dose, open-label, sequential ascending dose study to evaluate the safety and tolerance of pyridoxal-5′-phosphate, in an enteric-coated tablet, following a single dose of 250 mg, 750 mg, 1000 mg, and 4000 mg in healthy subjects under fasting conditions was conducted.

A total of 32 healthy, adult subjects signed the study-specific Informed Consent Form and were confined in the clinical study unit; of these subjects, 24 (6 subjects in each dose level; 3 males and 3 females) were dosed and were enrolled in the study; all of these enrolled subjects completed the study. Subjects were confined to the SFBC Anapharm Clinical Research Facility from at least 12 hours prior to drug administration until after the 24.0-hour post-dose blood draw.

Subjects enrolled in this study were members of the community at large. Subject screening procedures included informed consent, inclusion/exclusion check, demography, medical history, medication history, physical examination, height, weight, body mass index, a concomitant medication check, vital signs measurements (blood pressure, pulse rate, respiratory rate, and oral temperature), a 12-lead electrocardiogram (ECG), a urine drug screen, a urine pregnancy test (female subjects), hematology, biochemistry, urinalysis, and HIV and hepatitis testing. All participating subjects were judged eligible for the study when assessed against the inclusion and exclusion criteria.

All cohorts were sequentially dosed in an ascending fashion. Subsequent cohorts were dosed only after the completion of clinical part of the previous cohort and after revision, by the Sponsor and the Qualified Investigator, of the safety data.

Subjects were administered a single oral dose of study medication, as a 1×250 mg (Cohort 1), 3×250 mg (Cohort 2; total dose of 750 mg), 4×250 mg (Cohort 3; total dose of 1000 mg), or 16×250 mg (Cohort 4; total dose of 4000 mg) enteric-coated tablets.

After a supervised overnight fast of at least 10 hours, subjects were administered the medication as a single oral dose of 1, 3, 4, or 16 enteric-coated tablets containing 250 mg of P5P (total dose of 250mg, 750 mg, 1000 mg, or 4000 mg), with 300 mL of water. Subjects were dosed as specified in the protocol, and subsequently fasted for at least 4 hours. Subjects in Cohorts 2 to 4 did not receive their dose until the clinical part of the preceding dose level was completed, the safety data reviewed by the Principal Investigator and the Sponsor, and a decision taken to proceed or not with the next dose level.

Clinical laboratory tests (hematology, biochemistry, and urinalysis) were performed for each subject at the time of the screening and post-study procedures and prior to dosing.

The pharmacokinetic parameters to determine bioavailability for this study were: area under the concentration-time curve from time zero to time of last non-zero concentration (AUC₀₋ₜ), maximum observed concentration (Cₘₐₓ), time of observed Cₘₐₓ (Tₘₐₓ) and elimination half-life (equivalent to t_{1/2}).

P5P produced optimal effects when administered at 250 mg due to a directly proportional relationship between the dose administered and the variability of plasma P5P concentrations achieved in a subject (Table 1), most likely due to unknown enzymatic activities.

TABLE 1
Summary of baseline-corrected pharmacokinetic parameters
for the enteric-coated tablet

| | Dose | | | |
|---|---|---|---|---|
| | 250 mg | 750 mg | 1000 mg | 4000 mg |
| AUC₀₋ₜ | 2.6 ± 1.3 | 12.8 ± 15.4 | 5.9 ± 7.7 | 6.9 ± 7.4 |
| (μg · h/mL) | | | | |
| Cₘₐₓ (μg/mL) | 0.3 ± 0.2 | 3.9 ± 5.4 | 1.5 ± 2.3 | 1.7 ± 2.7 |
| Tₘₐₓ (h) | 3.2 ± 1.3 | 2.8 ± 0.9 | 2.9 ± 1.4 | 4.7 ± 3.5 |
| T_{1/2} (h) | 53.8 ± 41.9 | 27.9 ± 19.5 | 36.2 ± 30.6 | 16.5 ± 16.5 |

All of the patients proceeded to the highest dosage form, and none of the patients presented any significant side effects or evidence of toxicity. Thus P5P was found to be well tolerated in patients in dosages up to 4000 mg.

### EXAMPLE 2

### Treatment of Tardive Dyskinesia with P5P

A double blind, placebo controlled crossover study is conducted among patients with TD induced by the use of a neuroleptic medication. Patients are divided into 5 groups: control, Vitamin B6 treatment (400 mg), low dose of P5P (100 mg), medium dose of P5P (250 mg), and high dose of P5P (750 mg). All dosages are administered orally. Treatments are given daily.

All groups treated with P5P have improved scores on the subscales of the Extrapyramidal Symptom Rating Scale, more specifically in the parkinsonism and dyskinetic movement scales, as compared to either the control or vitamin B6 treated groups. Groups treated with medium or high dose of P5P exhibit significantly improved scores as compared to low dose group. Groups treated with medium and high dose of P5P do not exhibit significantly different scores to one another.

1-31. (canceled)

32. A method for the treatment of tardive dyskinesia comprising administering pyridoxal 5′-phosphate.

33. The method of claim 32 wherein the tardive dyskinesia is a result of use of schizophrenia medication.

34. The method of claim 33 wherein the schizophrenia medication is a neuroleptic.

35. The method of claim 32 wherein the pyridoxal 5′-phosphate is an oral administration of 100-4000 mg/day.

36. The method of claim 35 wherein the pyridoxal 5′-phosphate is between 100-750 mg/day.

37. The method of claim 35 wherein the pyridoxal 5′-phosphate is about 250 mg/day.

38. The method of claim 35 wherein the pyridoxal 5′-phosphate is provided in combination with a schizophrenia drug.

39. The method of claim 38 wherein the schizophrenia drug is a neuroleptic.

40. A kit comprising:
(a) a pharmaceutical preparation for oral administration comprising pyridoxal 5′-phosphate; and
(b) instructions for the administration of said preparation;
wherein said instructions specify that the preparation is to be administered daily to a patient for the treatment of tardive dysknesia.

41. The kit of claim 40 wherein the instructions further specify that the preparation should be administered in a dosage range between 100-4000 mg/day.

42. A pharmaceutical compound comprising pyridoxal 5′-phosphate and a schizophrenia drug.

43. The pharmaceutical compound of claim 42 wherein the schizophrenia drug is a neuroleptic.
